(19) United States
(12) Patent Application Publication
(10) Pub. No.: US 2021/0068864 A1
(43) Pub. Date: Mar. 11, 2021

(54) SURGICAL ACCESS SYSTEM

(71) Applicant: Tsalys Medical Technologies Ltd, Shenzhen (CN)

(72) Inventor: Lun Man Cheung, Tsing Yi (CN)

(73) Assignee: Tsalys Medical Technologies Ltd, Shenzhen (CN)

(21) Appl. No.: 16/965,821

(22) PCT Filed: Jan. 31, 2018
(86) PCT No.: PCT/CN2018/074679

§ 371 Date: Jul. 29, 2020

(51) Int. Cl. A61B 17/34

(52) U.S. Cl. CPC A61B 17/3423; A61B 2017/00407; A61B 2017/3447; A61B 17/3474

(57) ABSTRACT

A surgical access system, includes a gas insufflation device, a multi-instrument access platform and a surgical smoke evacuation device, wherein the gas insufflation device is connected to a connector of the multi-instrument access platform, and an outlet and an inlet of the multi-instrument access platform are respectively connected to an inflow tube and an outflow tube of the surgical smoke evacuation device.

### CROSS-REFERENCE TO RELATED APPLICATIONS

This application is a national stage filing under 35 U.S.C. § 371 of international application number PCT/CN2018/074679, filed Jan. 31, 2018, the content of which is incorporated herein by reference in its entirety.

### TECHNICAL FIELD

The disclosure relates to the field of surgical instruments, and in particular to a surgical access system.

### BACKGROUND

During transanal minimally invasive surgery (TAMIS) and transanal total mesorectal excision (TaTME), most of the operations are performed by entering rectum and lower colon through anus and anal canal. The transanal method provides many benefits for surgeons and patients, such as easier observation and easier access to an important site for dissection and/or resection, but mainly because it can increase the “incisal margin” (distance) with a cancer site, and evidences have shown that maintaining the incisal margin can significantly reduce cancer recurrence. This advantage alone has led to the increased use of the transanal surgery. However, an access system through which instrument enter a body cavity of human body has not been specially developed or designed to overcome the anatomical variation and physiological challenges brought by such an operation to benefit patients. Current access system, such as Applied Gelpoint Path, is mainly designed for an abdominal cavity technology, without considering the special technology and operating environment required for the transanal surgery. The use of the existing access system will limit the positioning and placement of surgical instruments, because such an access system is designed to be fixed with sutures and cannot be repositioned when the depth and position of a lesion in the rectum change or when the anal canal variation (length and diameter) of individual patients is obvious. Furthermore, tissue cannot be easily manipulated under an aseptic state in the limited space of the anal canal.

In addition to the existing limitations caused by the current designs of access system, expanding or maintaining the size of a surgical cavity by CO₂ via an insufflator is mainly designed for transabdominal surgery and conventional laparoscopic surgery, and in addition there is a lack of compatible and integrated surgical smoke removal apparatus, which further increases the difficulty of efficiently completing the transanal surgery technology and practice within a small surgical cavity.

The surgical cavity is inflated with gas such as carbon dioxide (CO₂) to inflate the rectal cavity. The expansion of inert gas forms a space in the cavity, which enables a surgeon to use laparoscopic surgical instruments and standard techniques for surgery under direct visualization within an anatomical structure. Many insufflators supply CO₂ intermittently or in a pulse form, and pressured pulse and pressure measurement are alternately performed to monitor and maintain constant pressure and cavity size. The pressure sampling frequency and inflation frequency of products from different manufacturers vary. In addition, the operation site is not a sealed cavity, and CO₂ will leak from the inflated operation area, resulting in unpredictable pressure drops. Furthermore, CO₂ is easily absorbed by a colorectal wall, thereby aggravating the pressure loss caused by leakage. CO₂ may leak from the system by a variety of factor, such as the length of the colorectal system, the dose absorbed by the small intestine/colorectal wall, and leakage through surgical instruments and tools passing in and out from the access routes. A variety of leakage paths lead to pressure loss, and the pulsed inflation flow to compensate for the reduction in pressure itself appears as bulge of the rectal wall. This phenomenon is further aggravated by the fact that compared with the large abdominal cavity usually seen during the laparoscopic surgery and the pneumoperitoneum (usually inflated with 1.5-3 liters of gas according to the patient's body size), the effective anal cavity for the first step of the TATME operation, as formed by sealing the rectal canal below a pathological site, is extremely small (about 250-500 ml). A modern insufflator is calibrated and designed for the intra-abdominal volume, pressure and gas flow. The insufflator continuously samples the intra-abdominal pressure and supplies extra gas to compensate for the normal leakage gas. However, the sampling rate of the modern insufflator is not enough to maintain a constant pressure in a small cavity like the rectum. The Boyle's law describes how a gas pressure tends to increase with the decrease of a container volume. Applying the Boyle's law to this problem can help more clearly understand this extra problem to be overcome in theory and practice. Because of the above factors, the operator will notice movement or a respiratory effect. That is, the rectal wall is moved with the inflation rhythm of the insufflator. The degree of movement of the rectal wall depends largely on the effective volume of the cavity, the quality of the insufflator and the rate of gas leakage from the cavity.

The movement of the rectal wall follows the pressure cycle of the insufflator: when the insufflator supplies CO₂, the rectal wall expands, and when the insufflator does not supply CO₂ (when it measures pressure), the rectal wall contracts. In TAMIS or other transanal operations that require benign or malignant pathological manipulation and treatment on or in the rectal wall, the movement of the rectal wall may make precision laparoscopic surgery more difficult.

When an electrosurgical instrument is used during the operation, another difficulty arises. Due to the nature of the device, a preformed electrode element used for tissue cutting dissection and coagulation during surgery will, at its tip generate heat through high frequency and high voltage electricity. These devices generate a surgical smoke plume which is a mixture of tissue, cellular and potentially virul particles and water at the operation site. Accumulation of the smoke plume may seriously reduce the visibility of the operation site, thereby significantly affecting the safety and effectiveness of the surgery. In a traditional laparoscopic surgery, the surgeon regularly opens and closes an entrance (puncture outfit) or a valve thereof to discharge the smoke plume into the atmosphere, or in some cases connects the entrance to a special smoke evacuation system, which effectively discharges the smoke plume from the abdomen into a specially designed and filtered container. Because the size of the anal cavity is relatively small, the accumulation of this smoke plume is more rapidly created when an electrosurgical technique is used, which is considered a seriously limiting factor to the wider application of the transanal TaTME or TAMIS techniques.

### SUMMARY

The disclosure provides a surgical access system in order to solve the problems of the surgical access system in the prior art.

In order to solve the above problem, the technical solution adopted by the disclosure is as follows.

There is provided a surgical access system, including a gas insufflation device, a multi-instrument access platform and a surgical smoke evacuation device; wherein the air blowing device is connected to a connector of the multi-instrument access platform, and an outlet and an inlet of the multi-instrument access platform are respectively connected to an inflow tube and an outflow tube of the surgical smoke evacuation device.

Preferably, the multi-instrument access platform includes an access platform cover or cap and an access tube, and the access platform cover or cap is covered on the access tube; the access platform cover or cap includes a semi-flexible rubber top cover and a rigid annular frame which are fixed together, at least three instrument channels are arranged on the semi-flexible rubber top cover, and the instrument channels are connected with the semi-flexible rubber top cover through a fold; the rigid annular frame is butted cooperatively with the access tube; the rigid annular frame is further provided with an integrally formed inflation inlet, and an additional outlet and inlet on a side of the access platform cover or cap opposite to the inflation inlet; and a variable ratchet is arranged on the access tube, and the variable ratchet is connected with a support ring.

Preferably, one end of the inlet leading to the access tube is connected with a smoke ring evacuation port; the inlet has a lumen diameter gradually decreasing along a direction of entering the access tube; and the outlet has a lumen diameter gradually increasing along the direction of entering the access tube.

Preferably, a rubber flange is arranged on the access tube, an integrally formed conical face is arranged on an inner side of the access platform cover or cap connected with the rigid annular frame, and the conical face is butted cooperatively with the rubber flange.

Preferably, the inflation inlet is a straight tube connected with an inflation valve and the connector connected with the gas insufflation device.

Preferably, the gas insufflation device is a CO₂ medical grade insufflator.

Preferably, the surgical smoke evacuation device includes a gas storage container which includes an inflow filter and an outflow filter which are respectively arranged at an inlet hole and/or an outlet hole, and the inflow filter and the outflow filter are respectively connected with the inflow tube and the outflow tube; and the gas storage container further includes a volume-variable chamber.

Preferably, the system further includes a pump arranged on the outflow tube connected with the outflow filter; the pump is powered by an integrated battery or a power supply; and the pump is a variable-speed pump.

Preferably, the inflow tube is connected to the outlet of the multi-instrument access platform, and the outflow tube is connected with the inlet of the multi-instrument access platform; or, the inflow tube is connected with an outlet of the gas insufflation device, and the outflow tube is connected with an inlet of a laparoscopic passage.

Preferably, the gas storage container includes at least one gas storage bag; the at least one gas storage bag is separated by a pressure-sealed zipper line or combined by a sliding zipper; and the gas storage container is made of medical-grade plastic, cellophane or a biodegradable plastic material.

The disclosure has the following beneficial effects: a surgical access system is provided, the depth of insertion of the access tube during an operation can be adjusted, and the angle at which an instrument enters a human body can be adjusted, so as to provide better positioning of the access tube according to the location of a lesion or to work more efficiently under different rectal conditions; meanwhile, better positioning of the instrument during an operation can be achieved, and if a tissue needs to be removed during surgery, the cover can be conveniently removed and installed with one hand; the smoke generated during a laparoscopic surgery can be removed, regardless of the size of the surgical cavity, without affecting the safety during operation of the gas insufflation device, thereby keeping the pneumoperitoneum stable while evacuating smoke during gas insufflation in a simple and cost-effective way.

### BRIEF DESCRIPTION OF THE DRAWINGS

FIG. 1 is a schematic structural diagram of a multi-instrument access platform in Embodiment one of the disclosure;

FIG. 2 is a schematic structural diagram of another multi-instrument access platform in Embodiment one of the disclosure;

FIG. 3 is a top view of an access platform cover or cap in Embodiment one of the disclosure;

FIG. 4 is a schematic diagram of the internal structure of the multi-instrument access platform in Embodiment one of the disclosure;

FIG. 5 is a schematic sectional view of the multi-instrument access platform in Embodiment one of the disclosure;

FIG. 6 is a sectional view of the multi-instrument access platform in Embodiment one of the disclosure;

FIG. 7 is a partial enlarged view of the sectional view of the multi-instrument access platform in Embodiment one of the disclosure;

FIG. 8 is a schematic structural diagram of a further multi-instrument access platform in Embodiment one of the disclosure;

FIG. 9 is a schematic diagram showing the use of a surgical smoke evacuation device in Embodiment two of the disclosure;

FIG. 10 is a schematic diagram showing the use of another surgical smoke evacuation device in Embodiment two of the disclosure;

FIG. 11 is a schematic structural diagram of a gas storage container in Embodiment two of the disclosure; and

FIG. 12 is a schematic structural diagram of a surgical access system in Embodiment three of the disclosure;

wherein, 1—access platform cover or cap, 2—access tube, 3—variable ratchet, 4—support ring, 5—sutural pore, 6—pressing latch, 7—instrument channel, 8—semi-flexible rubber top cover, 9—fold, 10—rigid annular frame, 11—inflation inlet, 12—inflation valve, 13—connector, 14—outlet, 15—inlet, 16—smoke ring evacuation port, 17—operation site, 18—pump motor, 19—inflow tube, 20—gas storage container, 21—inflow filter, 22—pump, 23—outflow filter, 24—outflow tube, 25—rubber flange, 26—conical face, 27—insufflator, 28—standard sterile flexible tube, 29—laparoscope, 30—cutting instrument, 31—electrosurgical electric knife head, 32—abdominal wall, 33—multi-instrument access platform, 34—gas flow direction, 35—human tissue, 36—surgical cavity, 37—gas storage bag, 38—zipper line, and 39—sliding zipper.

### DETAILED DESCRIPTION

The disclosure is described in detail hereafter by specific examples in connection with accompanying drawings, so as to help better understand the disclosure, but the following examples do not limit the scope of the disclosure. Moreover, it should be noted that the illustrations provided in the following embodiments only illustrate the basic concept of the disclosure in a schematic way, and the accompanying drawings only show the components related to the disclosure, instead of depicting according to the number, shape and size of the components in actual implementation. The shape, number and proportion of each component can be randomly changed in actual implementation, and the lay-out and modality of the components may be more complex.

### Embodiment One

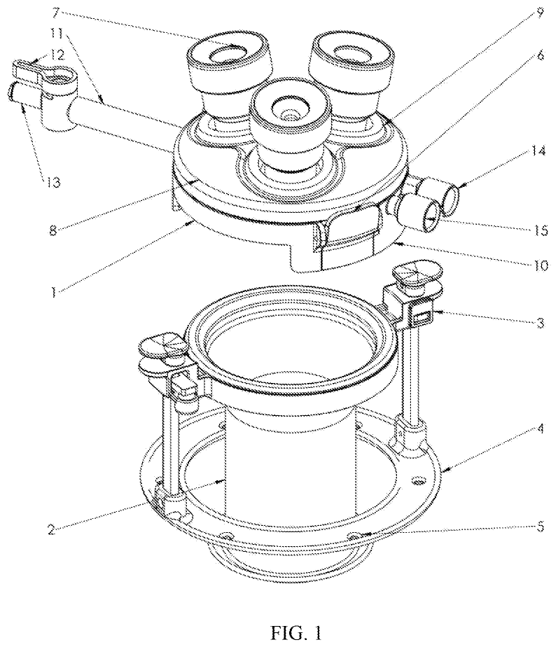

As shown in FIG. 1, the disclosure provides a multi-instrument access platform, which includes an access platform cover or cap 1 and an access tube 2. The access platform cover or cap 1 is covered on the access tube 2. The access platform cover or cap 1 includes a semi-flexible rubber top cover 8 and a rigid annular frame 10 which are fixed together. At least three instrument channels 7 are arranged on the semi-flexible rubber top cover 8. The instrument channels 7 are connected with the semi-flexible rubber top cover 8 through a fold 9. The rigid annular frame 10 is butted cooperatively with the access tube 2. The rigid annular frame 10 is further provided with an integrally formed inflation inlet 11, and an outlet 14 and an inlet 15 are provided at a side of the access platform cover or cap 1 opposite to the inflation inlet 11. A variable ratchet 3 is arranged on the access tube 2, and the variable ratchet 3 is connected with a support ring 4.

In an alternative embodiment of the disclosure, the support ring 4 is provided circumferentially with sutural pores 5. The access platform cover or cap 1 is closed on the access tube through a pressing latch 6. The rigid annular frame 10 is made of a hard rubber material. The inflation inlet 11 is a straight tube and is connected with an inflation valve 12 and a connector 13, and the connector 13 is connected with an insufflator.

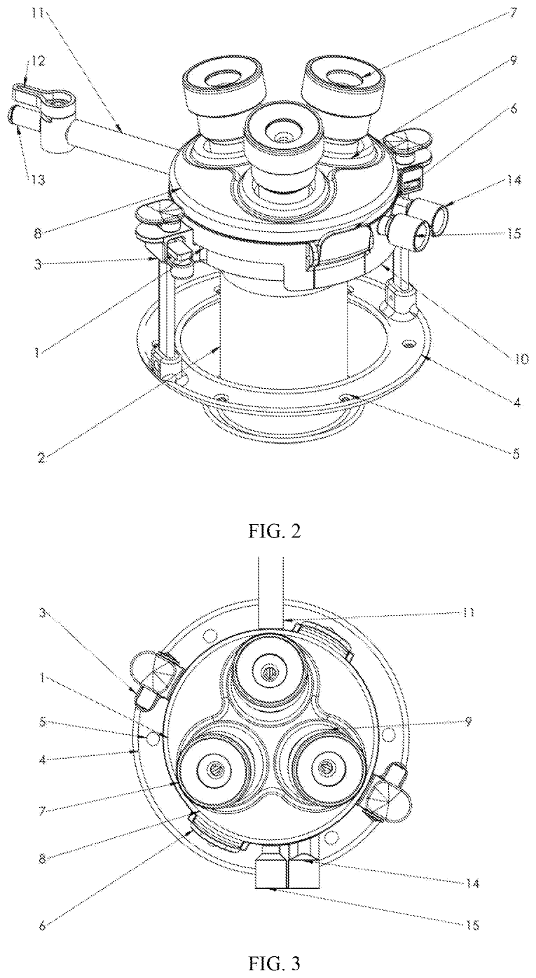

As shown in FIG. 2, it is the multi-instrument access platform when the access platform cover or cap 1 is closed on the access tube 2. Preferably, the access platform cover or cap 1 is sealed when closed.

As shown in FIG. 3, it is a top view of the access platform cover or cap 1. In this example, the semi-flexible rubber top cover 8 is provided with three instrument channels 7 thereon. It can be understood that four, five or even more instrument channels can be provided according to the needs of surgery, and the arrangement of the instrument channels on the semi-flexible rubber top cover is also not limited, and can be arranged according to actual needs.

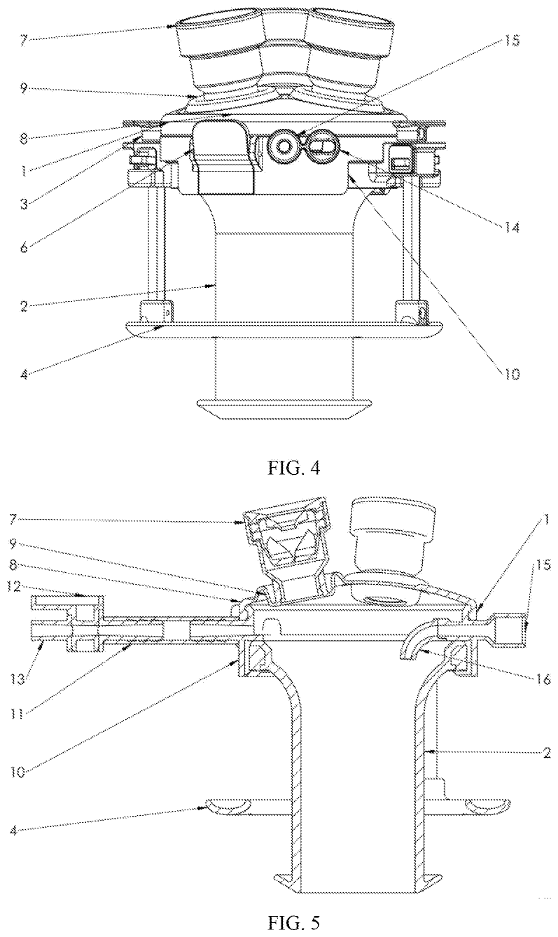

FIG. 4 is a schematic diagram of the internal structure of the multi-instrument access platform, showing the internal lumen connecting the inlet 15 and the outlet 14 of the inflation stabilization and smoke evacuation system. It should be noted that the inlet 15 has a the lumen diameter gradually decreasing along a direction of entering the access tube, so that the filtering gas used in the smoke cleaning of the access tube 2 and the operation site starts to accelerate, while the outlet 14 has a lumen diameter gradually increasing along the direction of entering the access tube, so as to reduce the flow resistance between the gas storage bag and the operation site, thereby making the surgical cavity and the gas storage bag respond as a whole.

FIG. 5 shows a cross section of the access platform and a smoke ring evacuation port 16 connected to the inlet 15 at a proximal end. The smoke ring evacuation port 16 guides the filtered pneumoperitoneum gas from an inflation and smoke evacuation circuit. The smoke ring evacuation port 16 guides a gas flow to circulate to a distal end, so as to “clear away” the smoke at and around the operation site at the distal end of the access tube 2. The smoke and residues are then removed through the outlet 14, and the gas is filtered by the inflation stabilization and smoke evacuation system for recycling. FIG. 5 also better shows the fold 9, which is designed to enable the instrument channels 7 to rotate with low resistance around a central fulcrum near the fold 9.

The gas flow evacuation from the smoke ring evacuation port 16 is especially used for promoting the gas flow to move along the axis of the access tube 2, thereby forming a CO₂ flow around the operation site. It is the result of careful experiments and tests that the smoke ring evacuation port is close to the access platform cover or cap 1. This position prevents the smoke ring evacuation port 16 from interfering with the instrument introduced through the instrument channel 7, and also prevents the evacuation from being too close to the tissue of a patient, which may otherwise dry or dehydrate the tissue in extreme cases. The smoke ring evacuation port 16 also has the additional function of heating and humidifying the circulating gas, such as heating the gas inside and outside the insufflator, in the gas storage container or in the pump.

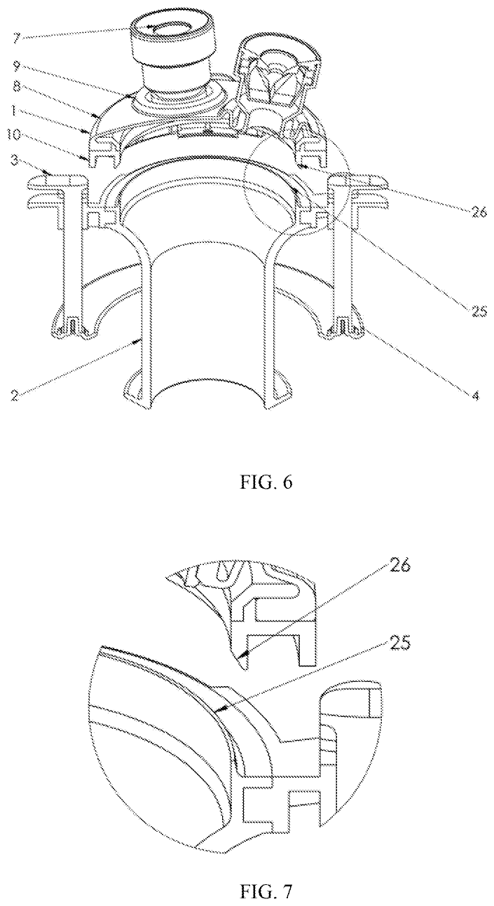

FIG. 6 shows a schematic sectional view of the multi-instrument access platform, and the enlarged part of the encircled section in the figure is shown in FIG. 7. As shown in FIGS. 6 and 7, the sealed connection between the access platform cover or cap 1 and the access tube 2 is realized by a rubber flange 25 provided on the access tube 2. An integrally formed conical face 26 is arranged on the inner side of the access platform cover or cap 1 connected with the rigid annular frame 10, and the conical face 26 is butted cooperatively with the rubber flange 25 for effective sealing. The conical face 26 and the rubber flange 25 co-act with the inner pressurized cavity to apply extra sealing pressure to the rubber flange 25, thereby maintaining the pneumoperitoneum, without using a gel or foam gasket to prevent leakage from the pressurized area.

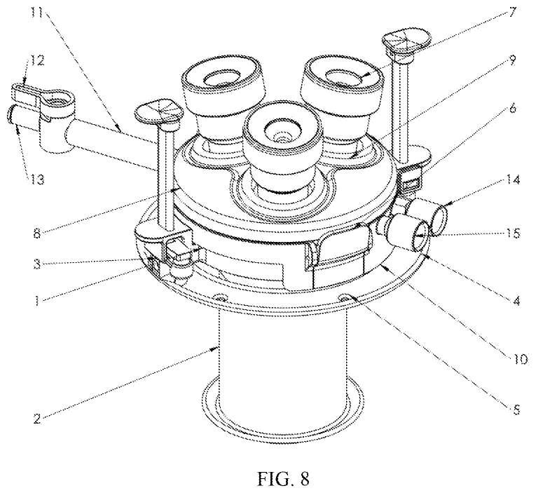

FIG. 8 shows that the access platform cover or cap 1 is closed on the access tube 2. Different from FIG. 2, the variable ratchet 3 is connected with the support ring 4 in a fully retracted state, and at this time the access tube 2 has the maximum insertion depth. In FIG. 2, the support ring 4 is in a fully-extended state, and at this time the access tube 2 has the minimum insertion depth. The support ring 4 can be extended or retracted according to anatomical requirements, sutural pores 5 are distributed circumferentially on the support ring 4, and then the support ring 4 is fixed at the patient through suture lines to provide stability for the instrument in surgery.

As shown in FIGS. 1-8, the multi-instrument access platform includes the access platform cover or cap 1, and the access tube 2 the height and entering depth of which are adjusted by a height/depth adjusting mechanism controlled by the variable ratchet mechanism 3. The access platform cover or cap 1 partially includes the semi-flexible rubber top cover 8, which carefully balances elasticity and rigidity, so that it is able to not only maintain its original position during inflation, but also allow a user to confirm the inflation process. It immobilizes the fulcrum position of the instrument passing through the instrument channel 7 and allows free inching/rotation of the instrument around the fulcrum. Three or more instrument channels 7 are arranged on the semi-flexible rubber top cover 8, and the connections between these instrument channels 7 and the semi-flexible rubber top cover 8 are specially designed folds 9, which provide more flexibility and less resistance for the movement and positioning of the instrument when it passes through the sealing valve of the instrument channel 7 compared with the planar modality of the semi-flexible rubber top cover 8. The semi-flexible rubber top cover 8 is fixed on the rigid annular frame 10, and the top end of access tube 2 is butted against the rigid annular frame 10. Meanwhile, the rigid annular frame 10 is provided with a plurality of integrally formed connecting tubes. The inflation inlet 11 is a straight tube connected with an inflation valve 12 and a connector 13, and the connector 13 is then connected with a traditional insufflator. An outlet 14 and an inlet 15 are arranged on the side opposite to the inflation inlet 11. The outlet 14 forms the outflow part of the inflation stabilization and smoke filtration system. The inlet 15 is used for the smoke evacuation circuit and inflation stabilization system described later in the specification.

The multi-instrument access platform provided by the disclosure can be applied to a system for the laparoscopic surgery or other operations. Compared with the access platform in the prior art, the multi-instrument access platform of the disclosure has the following advantages:

1. The multi-instrument access platform includes the support ring, so that the insertion depth of the access tube can be adjusted during the operation, thereby better positioning the access tube according to the lesion position or working more effectively under different rectal conditions (for the rectum that is longer or shorter compared with the general rectum).

2. The multi-instrument access platform can remove and reinstall the access platform cover or cap, rotate and reposition the access platform cover or cap and the instrument channel to provide better instrument positioning during surgery, and if a tissue needs to be removed during surgery, the cover can be conveniently removed and installed with one hand.

3. The joint between the semi-flexible rubber top cover and the instrument channel of the multi-instrument access platform is presented as a fold, and the instrument and visualization system (laparoscope) can move in a wider range with the fulcrum as the center, while keeping the fixed fulcrum for the instrument pivoting.

4. The sealing mechanism of the multi-instrument access platform prevents gas leakage in the pressurized space, the rubber flange in the sealing cover presses the conical face tightly, and the gas pressure in the access tube also exerts extra pressure on the rubber flange, which helps to form a completely sealed access tube.

5. The multi-instrument access platform includes an inflation inlet channel for inflation, and another inlet and an outlet that are specially used for forming a vision smoke removal and gas circulation system.

6. The outlet channel of the multi-instrument access platform is specially designed as a wide-hole channel, and the inlet channel is gradually narrowed. The narrow end of the inlet channel is connected with an evacuation port which is close to the access platform cover or cap and ejects to the distal end of the access tube, so as to guide the gas to flow in a direction parallel to the access tube and thus form a purging circulation circuit to transfer the gas, smoke and toxic substances from the operation site to a gas storage container equipped with special inflow and outflow filtration systems.

7. The multi-instrument access platform further includes a smoke ring evacuation port to guide the gas to flow from the proximal end of the access platform cover or cap to the distal operation site, so as to avoid the potential risk of tissue damage in the surgical cavity. This risk is caused by the drying and cooling effects when cold gas sweeps the human tissue at the operation site, such as drying and dehydration.

8. The multi-instrument access platform not only can control different insertion depths and has a rotatable and repositionable access platform cover or cap that can be easily disassembled, but also has three integrated ports on the access platform cover or cap for inflation, smoke evacuation and inflation stabilization.

### Embodiment Two

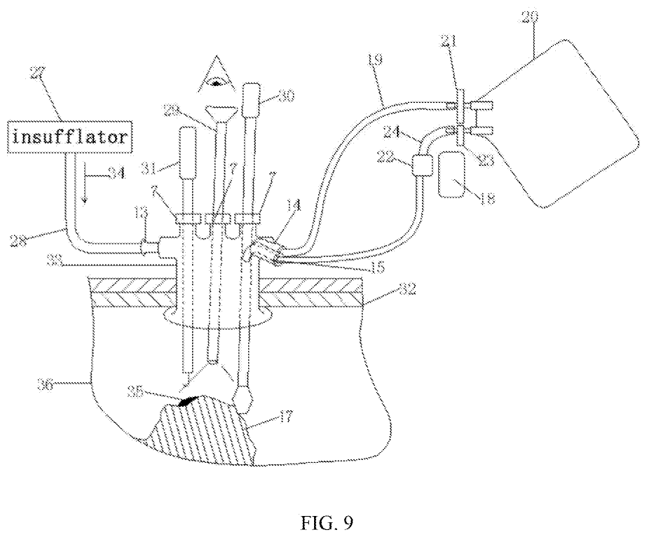

As shown in FIG. 9, the disclosure provides a surgical smoke evacuation device, which includes a gas storage container 20. The gas storage container 20 includes an inflow filter 21 and an outflow filter 23 which are respectively arranged at an inlet hole and/or an outlet hole, and the inflow filter 21 and the outflow filter 23 are respectively connected with an inflow tube 19 and an outflow tube 24. The gas storage container 20 also includes a volume-variable chamber.

In an alternative embodiment of the disclosure, the surgical smoke evacuation device includes a pump 22 arranged on the outflow tube 24 connected with the outflow filter 23. The pump 22 is powered by an integrated battery or a power supply. The pump 22 is a variable-speed pump.

As shown in FIG. 9, a surgical smoke evacuation device of the disclosure is shown in a schematic surgical sectional view of a typical surgical cavity 36. The figure shows an undergoing electrosurgical anatomy or a pathological operation of coagulating and embedding in the operation site 17. The insufflator 27 is connected to the connector 13 of the multi-instrument access platform 33 via a standard sterile flexible tube 28 to enlarge the surgical cavity, wherein the multi-instrument access platform 33 is fixed in an incision through the abdominal wall 32 of the patient. The gas from the insufflator 27 flows through the standard sterile flexible tube 28 in the direction indicated by the gas flow direction 34 to maintain a stable intra-abdominal pressure and provide space for visual observation of a human tissue 35 and the operation site 17, anatomy treatment and removal. Various surgical instruments and viewing apparatuses enter the surgical cavity 36 via the instrument channels 7 of the multi-instrument access platform 33. As described in the previous example, there are three or more instrument channels 7 in the multi-instrument access platform 33. A sealing valve is arranged in the instrument channel 7, and the purpose of the sealing valve is to enable the surgical instrument to pass through, be removed and move without loss of the pressure in the surgical cavity 36. A laparoscope 29 is placed in one instrument channel 7 for conveniently observing internal organs. In most cases, the laparoscope 29 is connected to a camera and an external screen for observation by a clinician. An electrosurgical electric knife head 31 is placed in another instrument channel 7 for cutting and coagulating the tissue by using high-frequency heat for cutting and coagulating. When the electrosurgical electric knife head 31 comes into contact with the tissue during operation, these instruments will form smoke and vaporize the tissue, which will blur the operator's vision and adhere to the laparoscope 29. Placed in one instrument channel 7 is a cutting instrument 30, or alternatively a grasping instrument for manipulating, retracting and moving the tissue. Once the surgical cavity is pressurized with the gas supplied by the insufflator 27, the gas will flow outward via the outlet 14 and then forward into the inflow filter 21 via the inflow tube 19 and into the gas storage container 20 via the inflow filter 21. The insufflator 27 will normalize the pressure in the gas storage container 20 to match the pressure in the surgical cavity 36, and the gas storage container 20 will expand. The insufflator 27 will measure the pressures of both the surgical cavity 36 and the gas storage container 20, and because the effective volume of the surgical cavity 36 is increased by increasing the external gas storage, the intra-abdominal pressure can be more effectively stabilized, and thus the influence described by the Boyle's Law can be eliminated. Therefore, the anatomical structure, especially the abdominal wall 32 and other flexible structures that may oscillate or swell, can be effectively stabilized, since the insufflator 27 maintains a stable pressure and compensates for pressure loss through gas leakage caused by instruments passing in and out of the instrument channels 7 and the physiological absorption of the gas.

The gas storage container 20 is provided with the outflow filter 23 for other gas flowing out from the inside. The gas enters the pump 22 embedded in the outflow tube 24 through a flexible tube via the outflow filter 23, and then enters the multi-instrument access platform 33 via a connecting piece. A pump motor 18 is connected to push the pump 22 so as to promote the gas flow into the operation site 17 and complete the circuitous flow, and meanwhile the gas containing smoke and harmful smoke plume in its circulation route flows towards the filtered gas storage container 20. The completion of the loop creates a stable gas circulation. The gas carrying smoke and harmful gas is filtered by the inflow filter 21 and the outflow filter 23 and then returned, thereby creating a clear line of sight and a smoke-free environment to complete the surgery. Combined with the stabilizing effect of the gas storage container 20, a stable operation site and instrument are created without bulging.

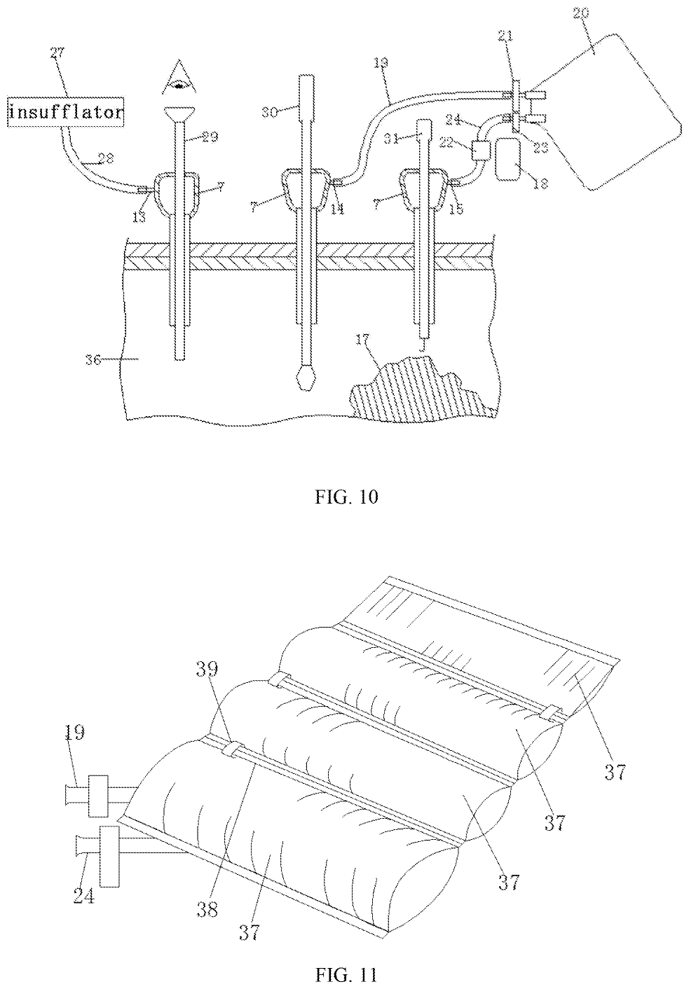

Referring now to FIG. 10, a more conventional laparoscopy method is shown: a standard laparoscopic trocar instrument channel 7 is used for entering the abdomen or surgical cavity. As with the conventional standard technology, the pneumoperitoneum is formed, so that the insufflator 27, which usually provides the gas of carbon dioxide, blows gas into the surgical cavity 36 via the standard sterile flexible tube 28 and is connected to a luer connector or connector 13 located on the outer cover of a laparoscopic orifice. Gas will enter and reach the operation site 17, and a pressure of 10-20 mmHg is usually set according to the patient's condition. Then, the insufflator 27 maintains a stable pressure as soon as possible to compensate for the pressure loss caused by leakage or absorption through other orifices and instruments, so as to prevent excessive pressure or excessive absorption of gas into surrounding tissues and organs. Generally, two or more additional laparoscopic instrument channels 7 are positioned so that the instruments can be easily contacted and directly observed, so as to start dissecting or electrotherapy on the operated tissues and lesions. The positions of the visualization laparoscope 29 and the electrosurgical electric knife head 31 or the grasping or cutting instrument 30 in this case will be different during the operation, wherein the instrument channel 7 is further fixed by puncturing at the operation site 17 to contact the lesion.

Particularly depending on the operation conditions, the surgeon can choose to connect only the blowing pressure stabilization, only the smoke evacuation circuit or both when noticed the bulging or movement of the tissue, specifically as follows.

Blowing stabilization: one end of the inflow tube 19 is connected to the outlet 14 of the instrument channel 7, and the other end is connected to the inflow filter 21 and enters the gas storage container 20. Combined with the volume of the gas storage container 20 and the volume of the operation site 17, the increased effective volume will stabilize the pneumoperitoneum. If smoke removal and stabilization are desired after blowing stabilization, the outflow filter 23 is connected to the outflow tube 24 having the pump 22 that is connected to an inlet connection for the inlet 15 of the instrument channel 7, and the pump motor 18 is activated to promote gas flow and complete a degassing loop, so that smoke and steam are transferred from the operation site 17 via the filtered and activated circulation system.

Now referring to FIG. 11 which is an illustration of a volume-expandable gas storage container 20. The volume-expandable gas storage container 20 is shown as connected to a filter in this case, wherein the inflow tube is denoted by 19 and the outflow tube is denoted by 24. The volume-variable gas storage container 20 may be made of various materials, such as medical-grade plastic, cellophane or biodegradable plastic materials, so as to facilitate treatment and avoid environmental pollution. It may further include at least one gas storage bag. The gas storage bag 37 is separated from an adjacent second gas storage bag 37 by a pressure-sealed zipper line 38 (which is opened by a sliding zipper 39) and sealed separately, so that the gas storage bag 37 has a certain volume at the beginning and gradually increases its effective volume as the zipper line is gradually opened. In this illustration, three gas storage bags 37 are shown as inflated, while the fourth gas storage bag 37 remains sealed with respect to the gas storage bags which have been opened and connected with each other. If the cavity of each gas storage bag has a volume of 500 mL, the cavity combined by three gas storage bags will represent and effectively increase a gas storage bag volume of 1.5 liters. It can be understood that this example should not be regarded as a limitation of the disclosure, but only lists one of the usage situations.

The purpose of the disclosure is to provide a surgical smoke evacuation device which can ensure stable pressure and minimum movement of the cavity or cavity wall during surgery, thereby creating a stable operation environment; while allowing the insufflator to maintain safe and effective flow of the insufflated gas in the cavity regardless of the size of the cavity and the leakage amount during surgery.

An energy source instrument used in the laparoscopic surgery, such as an electrocauterization device, a laser system and an ultrasonic scalpel, produce a gas by-product of aerosol, including viable and non-viable cellular substances. Such operation smoke will blur the operation area and have adverse effects on the human body. Most laparoscopic surgeons focus on maintaining a good line of sight in the operation area, and the most common method is releasing or discharging the smoke into the environment of the operating room. Some medical centers use filters for the surgical smoke, but these filters focus on removing the smoke to keep the surgeon's line of sight, rather than protecting the health of a person in a surgical facility.

In the conventional laparoscopic surgery, the surgeon will periodically discharge the smoke plume to the atmosphere by opening and closing the laparoscopic access orifice (trocar) or its valve, or in some cases the access orifice is connected to a special smoke removal system, which effectively removes the smoke plume from the abdomen into a specially designed and filtered container.

In a small cavity in the body, when the electrosurgical technique is used, this smoke accumulation will be further amplified and will seriously limit the completion of certain operations, because the surgeon's line of sight is damaged, and it is generally considered that this smoke accumulation seriously limits the wide application of TATME or TAMIS or other natural orifice transluminal endoscopic surgery techniques.

Another objective of the disclosure is to create a system, which can remove the smoke generated during the laparoscopic surgery, regardless of the size of the surgical cavity, without affecting the safety during operation of the insufflator as used.

Another objective of the disclosure is to solve the two independent but related problems of keeping the pneumoperitoneum stable during gas insufflation while evacuating smoke in a simple and cost-effective way.

### Embodiment Three

A surgical access system includes a gas insufflation device, an instrument access platform for entering an operation site as described in Embodiment one, and a surgical smoke evacuation device as described in Embodiment two. An outlet and an inlet of the access platform are connected with a gas storage bag or a gas storage container of the surgical smoke evacuation device, so that the inflation of the system is stabilized. The gas storage container promotes circulation of the smoke through a pump powered by an integrated battery or a power supply, and purges the visual path through a filtering system to prevent the recycling of potentially harmful particulate smoke. The gas insufflation device in the surgical access system is an insufflator, and more preferably a pulse insufflator.

In some embodiments, the access platform or the surgical smoke evacuation device in the system can function independently of each other. In one embodiment, the multi-instrument access platform provides an improved access path through a natural orifice or an artificial incision, so that the depth into the orifice or the incision can be adjusted and fixed as required, and the instruments and visualization equipment can be easily manipulated. For example, the fold on the multi-instrument access platform which is arranged around the instrument channel on the access platform cover or cap makes the laparoscope and the laparoscopic instruments (scissors, graspers and endoscopic surgical instruments) easier to operate, and a safe and easily controlled pressing latch is used for making the access platform cover or cap easy to fix and disassemble. Furthermore, in the system the access platform cover or cap can be rotated during placement or surgery, and can be adjusted flexibly by the user according to the operation condition, the patient's anatomy condition or the user's preference.

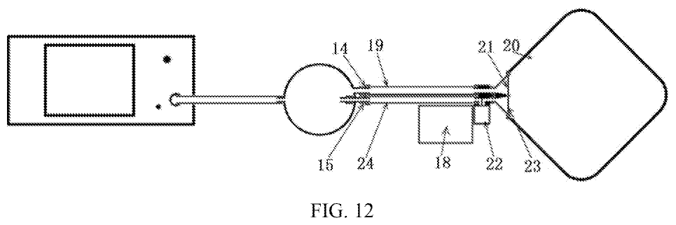

In another embodiment, any one of the multi-instrument access platform and the surgical smoke evacuation device in the surgical access system is connected with the traditional laparoscopic channel (sometimes called a puncture outfit) to independently maintain the pneumoperitoneum and/or evacuate the smoke. As shown in FIG. 12, by connecting the inflow tube 19 to the outlet of the conventional pulse insufflator and connecting the outflow tube 24 to the inlet of the puncture outfit connected with the conventional laparoscopic channel, the gas storage container 20 can also achieve complete stabilization of the pneumoperitoneum without the inflow filter 21, the outflow filter 23 and the pump 22.

In another embodiment, when connected to the insufflator and the luer connector or other connectors of the conventional laparoscopic channel (instead of connecting to the novel instrument channel), the gas storage container 20 together with the existing inflow tube 19, outflow tube 24, pump 22, inflow filter 21 and outflow filter 23 will form a smoke evacuation circuit for the conventional laparoscopic surgery.

In a further embodiment, the outlet 14 of the multi-instrument access platform is connected with the inflow tube 19 made of silica gel or other inflow tubes with similar soft inner cavities. The inflow tube 19 delivers gas and toxic smoke to the gas storage container 20. The main function of the gas storage container 20 is to inflate and pressurize the cavity by increasing and expanding the effective volume according to the principle explained in Embodiment one. An incoming carbonized gas containing microorganisms enters the inflow filter 21 through the inflow tube 19 to filter the gas and smoke plume, and then is delivered into the gas storage container 20. The filtered gas then passes through the outflow filter 23, and is delivered into the inlet 15 of the multi-instrument access platform through the outflow tube 24 made of silica gel under the action of a special pump 22 (shown as a single pump which can be disposable or reusable) powered by a battery or a power supply. Under the acceleration effect of the smoke ring evacuation port of the multi-instrument access platform, the gas removes the smoke around the operation site at the distal end of the access tube of the instrument access platform. In this process, through the smoke evacuation and filtration system of the surgical access system, the smoke generated by the electrosurgical operation can be continuously circulated, and meanwhile the pneumoperitoneum can be kept stable.

The above content is a further detailed description of the disclosure in connection with the specific preferred embodiments. It cannot be considered that the implementation of the disclosure is limited to the description. For those skilled in the art to which the disclosure belongs, on the premise of not departing from the conception of the disclosure, several equivalent substitutions or obvious modifications with the same performance or use can also be made, which should be regarded as belonging to the claimed scope of the disclosure.

1. A surgical access system, comprising an gas insufflation device, a multi-instrument access platform and a surgical smoke evacuation device; wherein the air blowing device is connected to a connector of the multi-instrument access platform, and an outlet and an inlet of the multi-instrument access platform are respectively connected to an inflow tube and an outflow tube of the surgical smoke evacuation device.

2. The surgical access system of claim 1, wherein the multi-instrument access platform comprises an access platform cover or cap and an access tube, and the access platform cover or cap is covered on the access tube;
the access platform cover or cap comprises a semi-flexible rubber top cover and a rigid annular frame which are fixed together, at least three instrument channels are arranged on the semi-flexible rubber top cover, and the instrument channels are connected with the semi-flexible rubber top cover through a fold; the rigid annular frame is butted cooperatively with the access tube;
the rigid annular frame is further provided with an integrally formed inflation inlet, and an outlet and an inlet on a side of the access platform cover or cap opposite to the inflation inlet; and
a variable ratchet is arranged on the access tube, and the variable ratchet is connected with a support ring.

3. The surgical access system of claim 2, wherein one end of the inlet leading to the access tube is connected with a smoke ring evacuation port; the inlet has a lumen diameter gradually decreasing along a direction of entering the access tube; and the outlet has a lumen diameter gradually increasing along the direction of entering the access tube.

4. The surgical access system of claim 2, wherein a rubber flange is arranged on the access tube, an integrally formed conical face is arranged on an inner side of the access platform cover or cap connected with the rigid annular frame, and the conical face is butted cooperatively with the rubber flange.

5. The surgical access system of claim 2, wherein the inflation inlet is a straight tube connected with an inflation valve and the connector connected with the gas insufflation device.

6. The surgical access system of claim 1, wherein the gas insufflation device is a CO₂ medical grade insufflator.

7. The surgical access system of claim 1, wherein the surgical smoke evacuation device comprises a gas storage container which comprises an inflow filter and an outflow filter which are respectively arranged at an inlet hole and/or an outlet hole, and the inflow filter and the outflow filter are respectively connected with the inflow tube and the outflow tube; and the gas storage container further comprises a volume-variable chamber.

8. The surgical access system of claim 7, further comprising a pump arranged on the outflow tube connected with the outflow filter; the pump is powered by an integrated battery or a power supply; and the pump is a variable-speed pump.

9. The surgical access system of claim 7, wherein the inflow tube is connected to the outlet of the multi-instrument access platform, and the outflow tube is connected with the inlet of the multi-instrument access platform; or, the inflow tube is connected with an outlet of the gas insufflation device, and the outflow tube is connected with an inlet of a laparoscopic passage.

10. The surgical access system of claim 7, wherein the gas storage container comprises at least one gas storage bag; the at least one gas storage bag is separated by a pressure-sealed zipper line or combined by a sliding zipper; and the gas storage container is made of medical-grade plastic, cellophane or a biodegradable plastic material.
